(19) United States
(12) Patent Application Publication
(10) Pub. No.: US 2017/0119653 A1
(43) Pub. Date: May 4, 2017

(54) ANTIPERSPIRANT OIL-IN-WATER EMULSIONS HAVING CROSS-LINKED SILICONE POLYMERS

(71) Applicant: Henkel AG & Co. KGaA, Duesseldorf (DE)

(72) Inventors: Thomas Doering, Dormagen (DE); Natascha Schevardo, Erkrath (DE)

(73) Assignee: Henkel AG & Co. KGaA, Duesseldorf (DE)

(21) Appl. No.: 15/335,519

(22) Filed: Oct. 27, 2016

(30) Foreign Application Priority Data
Oct. 28, 2015 (DE) 10 2015 221 058.8

(51) Int. Cl. A61K 8/89; A61K 8/26; A61K 8/28; A61Q 15/00; A61K 8/06

(52) U.S. Cl. CPC A61K 8/89; A61Q 15/00; A61K 8/062; A61K 8/28; A61K 8/26

(57) ABSTRACT

The present invention relates to antiperspirant cosmetic agents in the form of an O/W emulsion, which include a combination of an antiperspirant compound with a cross-linked silicone polymer in a weight ratio of 3:1 to 12:1. The previously mentioned combination leads to an improved antiperspirant effect of the O/W emulsions and to improved cosmetic properties, particularly reduced tackiness and textile soiling. The present invention also relates to a method for preventing and/or reducing the perspiration of the body by using the O/W emulsions and the use of at least one cross-linked silicone polymer in O/W emulsions to improve the antiperspirant effect of said emulsions.

### FIELD OF THE INVENTION

The present invention generally relates to an antiperspirant cosmetic agent in the form of an oil-in-water emulsion (O/W emulsion), which cosmetic agent includes at least one antiperspirant compound, selected from aluminum sesquichlorohydrate, phosphate-containing aluminum-zirconium salts, calcium-containing aluminum-zirconium salts, and mixtures thereof, in an aqueous phase and includes at least one oil phase having a cross-linked silicone polymer. The cosmetic agent according to the invention has a certain ratio of the antiperspirant compound to the cross-linked silicone polymer. The previously mentioned antiperspirant O/W emulsion leads to an improved antiperspirant effect and excellent cosmetic properties, particularly reduced tackiness and textile soiling.

Furthermore, the present invention relates to a non-therapeutic cosmetic method for preventing and/or reducing the perspiration of the body, wherein the antiperspirant cosmetic agent according to the invention in the form of an O/W emulsion is applied to the skin, particularly to the skin of the armpits, and remains there for at least 1 hour.

Finally, the present invention relates to the use of a cross-linked silicone polymer to improve the antiperspirant effect of O/W emulsions that include at least one antiperspirant compound, selected from the group of aluminum sesquichlorohydrate, phosphate-containing aluminum-zirconium salts, calcium-containing aluminum-zirconium salts, and mixtures thereof, in an aqueous phase.

### BACKGROUND OF THE INVENTION

The washing, cleaning, and care of one's own body is a basic human need, and modern industry is continually attempting to meet these human needs in a variety of ways. Especially important for daily hygiene is the lasting elimination or at least reduction of body odor and armpit moisture. Numerous specific deodorant or antiperspirant body care agents that have been developed for use in body regions having a high density of sweat glands, particularly in the armpit region, are known in the prior art. They are formulated in a wide range of application forms, for example as a powder, stick, aerosol spray, pump spray, liquid and gel roll-on application, cream, gel, and impregnated flexible substrates (deodorant wipes).

Cosmetic antiperspirants of the prior art include at least one antiperspirant salt. Aluminum(III) chloride, for example, is used as an antiperspirant salt. However, the use of aluminum(III) chloride in the amounts required for an adequate antiperspirant effect, usually more than 8 wt % with respect to the total weight of the agent, leads to poor skin compatibility of these agents. Furthermore, the use of aluminum-zirconium halides is known in the prior art. But such aluminum-zirconium halides are expensive and, in aqueous solutions, tend to form high-molecular-weight oligomeric and polymeric aluminum species that significantly reduce the effectiveness of these aluminum-zirconium salts in antiperspirants. However, the use of increased amounts of these antiperspirants to compensate the loss of effectiveness leads to considerably worse cosmetic properties, particularly a long-lasting tacky feeling on the skin and heavy staining of textiles.

For this reason, an attempt was made in the prior art to increase the effectiveness of the alkaline aluminum and aluminum-zinc halides by means of activation in order to reduce the total amount of antiperspirant active substance used and to improve the cosmetic properties in this way. For example, in documents EP 0308937 A2, EP 0183171 A2, U.S. Pat. No. 4,359,456 A, and EP 0191628 A2, alkaline aluminum and aluminum-zirconium halides that have been obtained by means of a thermal treatment are described. The heat-treated activated alkaline aluminum and aluminum-zirconium halides have a smaller proportion of high-molecular-weight species in comparison with untreated alkaline aluminum and aluminum-zirconium halides and therefore an increased antiperspirant effect.

Furthermore, the effectiveness of alkaline aluminum and aluminum-zirconium halides can be increased by integrating organic acids, phosphate ions, and calcium ions. For example, in documents U.S. Pat. No. 3,542,919 A, U.S. Pat. No. 3,553,316 A, U.S. Pat. No. 3,991,176 A, WO 2005/092795 A1, and U.S. Pat. No. 8,124,059 A, methods for producing such stabilized aluminum and aluminum-zirconium halides that have a higher proportion of short-chain species and are in activated form are disclosed.

However, the aforementioned activated alkaline aluminum and aluminum-zirconium halides of the prior art still have the disadvantage that the antiperspirant effect decreases with increasing storage duration and/or if protic solvents are used. The reason for this is that, in aqueous solution and in the typically used pH range of pH 4 to pH 7, these halides tend to form polymeric aluminum and/or zirconium complexes (referred to as Al complexes and Zr complexes below) having hydroxide and oxide bridges between the aluminum and/or zirconium ions, said complexes having a high molecular weight and a small antiperspirant effect.

Therefore, there is a need for antiperspirant cosmetic agents that have no significant decrease in the antiperspirant effect even during long periods of storage and/or if high amounts of protic solvents are used. Furthermore, the antiperspirants should be economical to produce and have good cosmetic properties, particularly reduced tackiness and staining of textiles and good skin compatibility.

The present invention addressed the problem of providing an antiperspirant cosmetic agent that avoids or at least diminishes the disadvantages of the prior art and in the case of which no significant decrease in the effectiveness of antiperspirant aluminum salts occurs during long periods of storage and/or in the presence of protic solvents. Furthermore, these cosmetic agents should be economical to produce and have good cosmetic properties, particularly reduced tackiness and staining of textiles and good skin compatibility.

Surprisingly, it has now been found that, by combining certain antiperspirant compounds with cross-linked silicone polymers, antiperspirant O/W emulsions having no significant decrease in the antiperspirant effect of these compounds during long periods of storage can be obtained. Because the decrease in the effectiveness of these antiperspirant compounds is avoided, reduced amounts can be used without negatively affecting the antiperspirant effectiveness. The use of reduced amounts of the antiperspirant compound leads to improved cosmetic properties, particularly reduced tackiness on the skin, reduced staining of textiles, and improved skin compatibility.

Furthermore, other desirable features and characteristics of the present invention will become apparent from the subsequent detailed description of the invention and the appended claims, taken in conjunction with this background of the invention.

### BRIEF SUMMARY OF THE INVENTION

An antiperspirant cosmetic agent in the form of an O/W emulsion, including at least one aqueous phase, comprising at least one antiperspirant compound, selected from aluminum sesquichlorohydrate, phosphate-containing aluminum-zirconium salts, calcium-containing aluminum-zirconium salts, and mixtures thereof; and at least one oil phase, comprising at least one cross-linked silicone polymer, wherein the weight ratio of the at least one antiperspirant compound in the aqueous phase to the at least one cross-linked silicone polymer in the oil phase is 3:1 to 12:1.

The use of at least one cross-linked silicone polymer to improve the antiperspirant effect of O/W emulsions that include at least one antiperspirant compound, selected from the group of aluminum sesquichlorohydrate, phosphate-containing aluminum-zirconium salts, calcium-containing aluminum-zirconium salts, and mixtures thereof, in an aqueous phase.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present invention relates to an antiperspirant cosmetic agent in the form of an O/W emulsion, containing
a) at least one aqueous phase, comprising at least one antiperspirant compound, selected from aluminum sesquichlorohydrate, phosphate-containing aluminum-zirconium salts, calcium-containing aluminum-zirconium salts, and mixtures thereof,
b) at least one oil phase, comprising at least one cross-linked silicone polymer, wherein the weight ratio of the at least one antiperspirant compound in the aqueous phase to the at least one cross-linked silicone polymer in the oil phase is 3:1 to 12:1.

According to the invention, the term “antiperspirant” is understood to mean the lessening or reduction of the perspiration of the sweat glands of the body.

Furthermore, in the sense of the present invention, the term “phosphate-containing aluminum-zirconium salt” is understood to mean a chemical compound that is constructed of positively charged ions (also called cations) in the form of aluminum and zirconium and negatively charged ions (also called anions) in the form of halides, particularly chlorides, and hydroxides and that additionally includes phosphate ions (PO₄³⁻).

In addition, according to the invention, the term “calcium-containing aluminum-zirconium salts” is understood to mean chemical compounds that are constructed of positively charged ions (also called cations) in the form of aluminum and zirconium and negatively charged ions (also called anions) in the form of halides, particularly chlorides, and hydroxides and that additionally include calcium ions (Ca²⁺).

In the context of the present invention, the term “cross-linked silicone polymers” should be understood to mean silicone polymers that have linking of the silicone polymer chains by direct covalent bonding or by bridging molecule fragments, which are covalently bonded to the silicone polymer chains. Therefore, cross-linked silicone polymers in the sense of the present invention have a network formed by covalent chemical bonds.

Furthermore, the term “fatty acid”, as it is used in the context of the present invention, should be understood to mean aliphatic carboxylic acids that have unbranched or branched carbon residues having 4 to 40 carbon atoms. The fatty acids used in the context of the present invention can be naturally occurring or synthetically produced fatty acids. Furthermore, the fatty acids can be mono- or polyunsaturated.

Finally, in the context of the present invention, the term “fatty alcohol” should be understood to mean aliphatic, monovalent, primary alcohols that have unbranched or branched hydrocarbon residues having 4 to 40 carbon atoms. The fatty alcohols used in the context of the invention can also be mono- or polyunsaturated.

In this document, the specification of wt % relates to the total weight of the antiperspirant cosmetic agent according to the invention in the form of an O/W emulsion, unless otherwise indicated.

The antiperspirant cosmetic agent according to the invention in the form of an O/W emulsion includes, as a first essential constituent, an aqueous phase a) having at least one antiperspirant compound, selected from aluminum sesquichlorohydrate, phosphate-containing aluminum-zirconium salts, calcium-containing aluminum-zirconium salts, and mixtures thereof.

The use of phosphate-containing antiperspirant aluminum-zirconium salts that include a certain total amount of phosphate, particularly phosphate ions (PO₄³⁻), is advantageous according to the invention. Therefore, it is preferred in the context of the present invention if the at least one phosphate-containing antiperspirant aluminum-zirconium salt includes phosphate, particularly phosphate ions (PO₄³⁻), in a total amount of 0.01 to 1.0 wt %, preferably 0.05 to 0.8 wt %, preferably 0.07 to 0.5 wt %, particularly 0.1 to 0.3 wt %, with respect to the total weight of the phosphate-containing antiperspirant aluminum-zirconium salt. According to the invention, the total amount of the phosphate is preferably understood to mean the total amount of phosphate ions, calculated as PO₄³⁻. The use of phosphate, particularly phosphate ions (PO₄³⁻), leads to improved stability of the aluminum-zirconium salts, particularly to reduced formation of high-molecular-weight polymers having only a small antiperspirant effect during storage. Therefore, these salts have an improved antiperspirant effect. The phosphate ions (PO₄³⁻) can be introduced, for example, during the production of the aluminum-zirconium salts by adding phosphoric acid or salts thereof, such as sodium phosphate and potassium phosphate, to aluminum-zirconium salts. To produce the antiperspirant O/W emulsions according to the invention, the phosphate-containing aluminum-zirconium salts and further constituents of the aqueous phase a) are heated to temperatures of 60 to 90° C., mixed with an oil phase b) likewise heated to a temperature of 60 to 90° C., and homogenized for a time period of 5 to 15 minutes. Then any further ingredients are added and the O/W emulsion is slowly cooled to a temperature of 20 to 25° C. while stirred.

With regard to the antiperspirant effect and the improved cosmetic properties, it is advantageous if the phosphate-containing aluminum-zirconium salts have certain molar ratios of phosphate, particularly phosphate ions (PO₄³⁻), to zirconium. Therefore, it is advantageous according to the invention if the at least one phosphate-containing antiperspirant aluminum-zirconium salt has a molar ratio of phosphate, particularly phosphate ions (PO₄³⁻), to zirconium of 0.001:1 to 0.5:1, preferably 0.005:1 to 0.4:1, preferably 0.01:1 to 0.3:1, particularly 0.02:1 to 0.151:1. The use of phosphate-containing antiperspirant aluminum-zirconium salts that have the previously indicated molar ratios of phosphate, particularly phosphate ions (PO₄³⁻), to zirconium leads to an improved antiperspirant effect and excellent cosmetic properties, particularly reduced tackiness on the skin and reduced staining of textiles, in combination with cross-linked silicone polymers in the oil phase.

If phosphate-containing and/or calcium-containing antiperspirant aluminum-zirconium salts are used in the context of the present invention, it has proven advantageous if they have a certain molar ratio of aluminum to zirconium. Preferred embodiments of the antiperspirant cosmetic agents according to the invention are therefore characterized in that each of the at least one phosphate-containing and/or calcium-containing aluminum-zirconium salt has a molar ratio of the sum of aluminum and zirconium (Al+Zr) to chloride of 0.3:1 to 2.5:1, preferably 0.5:1 to 2.4:1, preferably 0.7:1 to 2.3:1, particularly 0.9:1 to 2.1:1.

In the context of the present invention, the phosphate-containing and/or calcium-containing antiperspirant aluminum-zirconium salt used can additionally include the amino acid glycine. Preferred embodiments of the antiperspirant cosmetic agents according to the invention are therefore characterized in that each of the at least one phosphate-containing and/or calcium-containing aluminum-zirconium salt additionally includes glycine in a total amount of 2.0 to 8.0 wt %, preferably 3.0 to 7.0 wt %, preferably 3.5 to 6.5 wt %, particularly 4.0 to 6.0 wt %, with respect to the total weight of the phosphate-containing or calcium-containing antiperspirant aluminum-zirconium salt. In this context, it is also preferred if the molar ratio of glycine to zirconium in the previously mentioned salts is 0.5:1 to 1.5:1.

The use of calcium-containing antiperspirant aluminum-zirconium salts that include a certain total amount of calcium, particularly calcium ions (Ca²⁺), is advantageous according to the invention. Therefore, it is preferred in the context of the present invention if each of the at least one calcium-containing aluminum-zirconium salt includes calcium, particularly calcium ions (Ca²⁺), in a total amount of 0.1 to 6.0 wt %, preferably 0.2 to 3.0 wt %, preferably 0.3 to 1.0 wt %, particularly 0.4 to 0.7 wt %, with respect to the total weight of the calcium-containing aluminum-zirconium salt. According to the invention, the total amount of the calcium is preferably understood to mean the total amount of calcium ions, calculated as Ca²⁺. The use of calcium, particularly calcium ions (Ca²⁺), leads to improved stability of the aluminum-zirconium salts, particularly to reduced formation of high-molecular-weight polymers having only a small antiperspirant effect during storage. Therefore, these salts have an improved antiperspirant effect. The calcium ions (Ca²⁺) can be introduced, for example, during the production of the aluminum-zirconium salts by adding calcium chloride or calcium oxide. To produce the antiperspirant O/W emulsions according to the invention, the calcium-containing aluminum-zirconium salts and further constituents of the aqueous phase a) are heated to temperatures of 60 to 90° C., mixed with an oil phase b) likewise heated to a temperature of 60 to 90° C., and homogenized for a time period of 5 to 15 minutes. Then any further ingredients are added and the O/W emulsion is slowly cooled to a temperature of 20 to 25° C. while stirred.

The at least one phosphate-containing antiperspirant aluminum and/or aluminum-zirconium salt (i) is preferably used in certain amount ranges. Therefore, it is preferred in the context of the present invention if the at least one antiperspirant compound includes in a total amount of 5.0 to 14 wt %, preferably 6.0 to 14 wt %, preferably 7.0 to 13 wt %, particularly 10 to 12 wt %, with respect to the total weight of the antiperspirant cosmetic agent. The previously indicated total amount relates to all antiperspirant compounds, particularly of the aluminum sesquichlorohydrate and/or of the phosphate-containing aluminum-zirconium salt and/or of the calcium-containing aluminum-zirconium salt, included in the cosmetic agent according to the invention. The total amount of the antiperspirant compound is calculated without taking into account bonded water of crystallization and any ligands, such as glycine. Because of the improved antiperspirant effect due to combination with the at least one cross-linked silicone polymer in the oil phase, the amount of antiperspirant compound can be reduced to achieve antiperspirant performance comparable to that of cosmetic agents of the prior art that do not have this combination. The reduction of the amount of the antiperspirant compound leads to improved cosmetic properties, particularly reduced tackiness on the skin, reduced staining of textiles, and improved skin compatibility.

It is advantageous according to the invention if the aqueous phase a) includes water in certain amount ranges with respect to the total weight of the aqueous phase a). Preferably, the aqueous phase a) includes water in a total amount of 55 to 94 wt %, preferably 60 to 92 wt %, preferably 65 to 90 wt %, particularly 70 to 85 wt %, with respect to the total weight of the aqueous phase a).

In addition, it is advantageous according to the invention if the O/W emulsion according to the invention includes water in certain amount ranges with respect to the total weight of the O/W emulsion. According to the invention, it is therefore preferred if the antiperspirant cosmetic agent includes the aqueous phase a) in a total amount of 60 to 90 wt %, preferably 65 to 85 wt %, preferably 70 to 85 wt %, particularly 75 to 85 wt %, with respect to the total weight of the antiperspirant cosmetic agent.

It can be preferred that a thickener is added to the aqueous phase a) to set a desired viscosity of the O/W emulsion. This is advantageous in order to apply and distribute the O/W emulsions well at the location of application on the one hand, but on the other hand to set a sufficient viscosity so that the O/W emulsions remain at the location of action, particularly under the armpits, during the exposure time and do not run or are not excessively transferred to the clothing. Therefore, it is preferred in the context of the present invention if the aqueous phase a) additionally includes at least one non-ionic associative polymer, preferably a non-ionic associative polyurethane/polyether, particularly a steareth-100/PEG-136/HDI copolymer, in a total amount of 0.01 to 4.0 wt %, preferably 0.1 to 3.0 wt %, preferably 0.2 to 2.0 wt %, particularly 0.3 to 1.0 wt %, with respect to the total weight of the antiperspirant cosmetic agent. The term “non-ionic associative polymers” is understood to mean polymers that include hydrophobic terminal or side groups in addition to hydrophilic groups, such as polyoxyethylene groups, and bear neither anionic nor cationic or cationizable functional groups. The hydrophobic molecule part is preferably a hydrocarbon chain having 8 to 28 carbon atoms, which hydrocarbon chain can be saturated or unsaturated, linear or branched. This C₈-C₂₈-alkyl chain is especially preferably linear. The steareth-100/PEG-136/HDI copolymer especially preferably used according to the invention can be obtained by reacting the monomers steareth-100, PEG-136, and 1,6-hexamethylene diisocyanate (HDI). Such associative polymers not only are used as thickeners but also have an emulsifying effect and therefore can stabilize the O/W emulsion according to the invention.

The antiperspirant cosmetic agent includes, as a second essential constituent, an oil phase b) having at least one cross-linked silicone polymer.

In the context of the present invention, it is preferred if the cross-linked silicone polymer is selected from the group of non-ionic cross-linked silicone polymers, cationic cross-linked silicone polymers, and mixtures thereof. The use of such cross-linked silicone polymers in combination with the previously indicated antiperspirant compound has proven especially advantageous with regard to the antiperspirant effect and the reduced staining of textiles.

In this context, it is preferred that non-ionic cross-linked silicone polymers consisting of siloxane chains cross-linked with each other by alkyl groups are used. Such silicone polymers can be obtained, for example, by cross-linking siloxanes having at least two SiH groups within the polymer chain with compounds having at least two unsaturated alkyl groups at the ends of said compounds by using a hydrosilylation catalyst, particularly a platinum, iridium, rhodium, and palladium catalysts. Furthermore, such silicone polymers can be obtained by reacting a siloxane having, on at least two silicon atoms within the polymer chain, an allyl group having a terminal double bond with a siloxane containing at least two SiH groups within the polymer chain by using a hydrosilylation catalyst. Preferred embodiments are therefore characterized in that a cross-linked silicone polymer having at least one structural unit of formula (Ia) and at least one structural unit of formula (IIa) and at least one structural unit of formula (IIIa) is included as a non-ionic cross-linked silicone polymer,

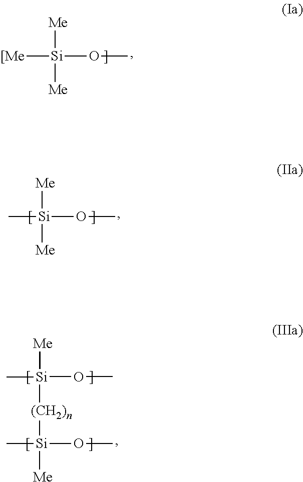

wherein
n represents integers from 2 to 10, preferably from 4 to 8, particularly 6. Such non-ionic cross-linked silicone polymers are known under the INCI name Dimethicone Crosspolymer and are available, for example, from Dow Corning under the trade name Dow Corning 7-3101 Elastomer Blend HIP Emulsion.

Furthermore, it can be preferred in this context that a non-ionic cross-linked silicone polymer, the siloxane chains of which are cross-linked to each other by alkoxy groups, is used. Such silicone polymers can be obtained, for example, by cross-linking siloxanes having at least two SiH groups within the polymer chain with alkoxy compounds having at least two unsaturated alkyl groups at the ends of said alkoxy compounds by using a hydrosilylation catalyst, particularly a platinum, iridium, rhodium, and palladium catalysts. Furthermore, such silicone polymers can be obtained by reacting a siloxane having, on at least two silicon atoms within the polymer chain, an alkoxy group having a terminal double bond with a siloxane containing at least two SiH groups within the polymer chain by using a hydrosilylation catalyst. In the context of this embodiment, it is therefore preferred if a cross-linked silicone polymer at least one structural unit of formula (Ib) and at least one structural unit of formula (Ith) and at least one structural unit of formula (IIIb) and at least one structural unit of formula (IVb) is included as a non-ionic cross-linked silicone polymer,

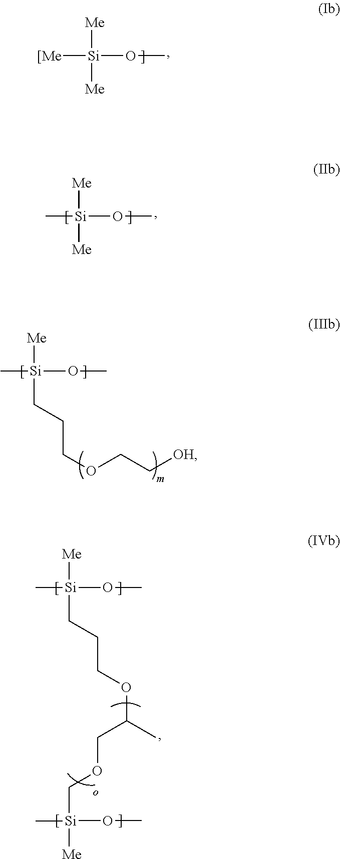

wherein
m represents integers from 6 to 16, preferably from 8 to 14, particularly from 10 to 14, and o represents integers from 10 to 30, preferably from 14 to 26, particularly from 18 to 22. Such non-ionic silicone polymers are known under the INCI name PEG-12 Dimethicone/PPG-20 Crosspolymer and are available, for example, from Dow Corning under the trade name Dow Corning EL-7040 Hydro Elastomer Blend.

Furthermore, non-ionic cross-linked silicone polymers cross-linked by alkyl groups can also be used. Such silicone polymers can be produced, for example, by cross-linking a silicone polymer having at least two free SiH groups with a silicone polymer having at least two alkenyl groups by using a previously described hydrosilylation catalyst. It can therefore be advantageous in the context of the present invention if a cross-linked silicone polymer having at least one structural unit of formula (Ic) and at least one structural unit of formula (IIc) and at least one structural unit of formula (IIIc) is included as a non-ionic cross-linked silicone polymer,

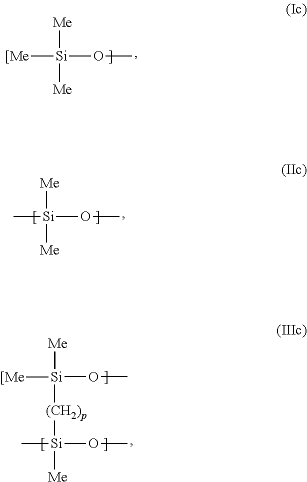

wherein
p represents integers from 2 to 6, preferably from 2 to 4, particularly 2. Such non-ionic silicone polymers are known under the INCI name Dimethicone/Vinyl Crosspolymer and are available, for example, from Dow Corning under the trade name Dow Corning PF-2520 Elastomer Emulsion or 9509 Silicone Elastomer Suspension.

The use of cationic cross-linked silicone polymers is also possible in the context of the present invention. Such silicone polymers can be obtained, for example, by reacting amino-functionalized siloxanes with epoxidized siloxanes and epoxide-containing quaternary ammonium compounds. It can therefore be preferred according to the invention if a cross-linked silicone polymer having at least one structural unit of formula (Id) and at least one structural unit of formula (IId) and at least one structural unit of formula (IIId) and at least one structural unit of formula (IVd) is included as a cationic cross-linked silicone polymer,

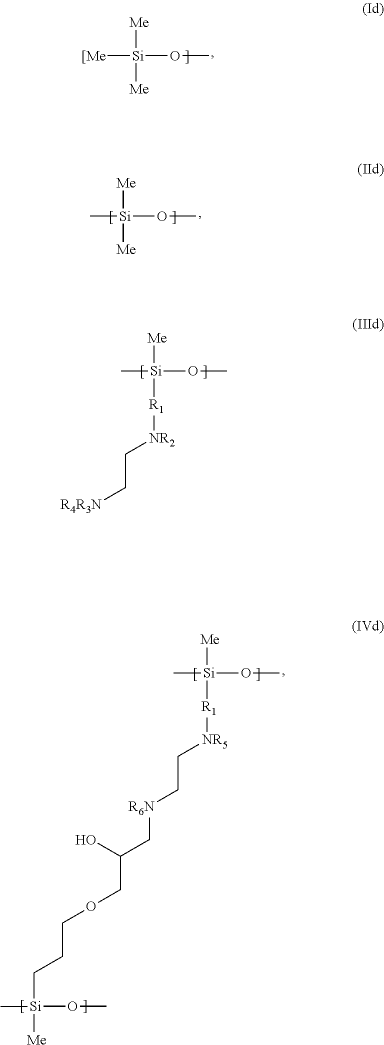

wherein
R₁ represents —CH₂—CH(CH₃)—CH₂— or —CH(CH(CH₃)₂)—,
R₂ to R₄ represent, independently of each other, hydrogen or —CH₂—CH(OH)—CH₂—N⁺(Me)₃Cl⁻, and
R₅ and R₆ represent, independently of each other in each case, hydrogen, —CH₂—CH(OH)—CH₂—N⁺(Me)₃Cl⁻, or —CH₂—CH(OH)—CH₂—O—(CH₂)₃—Si(Me)O_{2/2}. Here, the residue Si(Me)O_{2/2} means that a methyl residue and a further residue, for example a siloxane residue, are located on this silicon atom. Such silicone polymers are known under the INCI name Silicone Quaternium-16/Glycidoxy Dimethicone Crosspolymer and are available, for example, from Dow Corning under the trade name Dow Corning CE-7080 Smart Style.

It is preferred in the context of the present invention if the cross-linked silicone polymers used have certain particle sizes. Preferred embodiments of the present invention are therefore characterized in that the at least one cross-linked silicone polymer has an average particle size D₅₀ of 0.2 to 20 μm, preferably 1 to 15 μm, preferably 3 to 12 μm, particularly 4 to 11 μm. The average particle size D₅₀ of the cross-linked silicone polymers can be determined, for example, by means of dynamic light scattering (DLS) (Racles C. et. al; “On the feasibility of chemical reactions in the presence of siloxane-based surfactants”; Colloid and Polymer Science, 2009, 287, pages 461 to 470).

The at least one cross-linked silicone polymer is preferably used in certain amount ranges. Therefore, it is advantageous according to the invention if the antiperspirant cosmetic agent includes the at least one cross-linked silicone polymer in a total amount of 0.4 to 5.0 wt %, preferably 0.5 to 4.5 wt %, preferably 0.6 to 4.0 wt %, particularly 0.8 to 3.5 wt %, with respect to the total weight of the antiperspirant cosmetic agent. The previously indicated total amount relates to all the cross-linked silicone polymers included in the cosmetic agent according to the invention, particularly to the silicone polymers having structural units of formulas (Ia), (IIa), and (IIIa) and/or silicone polymers having structural units of formulas (Ib), (IIb), (IIIb), and (IVb) and/or silicone polymers having structural units of formulas (Ic), (IIc), and (IIIc) and/or silicone polymers having structural units of formulas (Id), (IId), (IIId), and (IVd). The use of the previously indicated total amounts of the cross-linked silicone polymer, particularly of the previously indicated preferred non-ionic and cationic cross-linked silicone polymers, leads to an improved antiperspirant effect of the O/W emulsions according to the invention and to improved cosmetic properties, particularly reduced tackiness on the skin and reduced staining of textiles, in combination with the antiperspirant compound.

According to the invention, the at least one antiperspirant compound in the aqueous phase a) must be used in a weight ratio to the cross-linked silicone polymer in the oil phase b) of 3:1 to 12:1 to achieve an excellent antiperspirant effect and good cosmetic properties. This effect can be improved further if these substances are used in narrower weight ratios. Preferred embodiments of the antiperspirant cosmetic agents according to the invention are therefore characterized in that the antiperspirant cosmetic agent has a weight ratio of the at least one antiperspirant compound in the aqueous phase a) to the cross-linked silicone polymer in the oil phase b) of 3:1 to 11:1, preferably 3:1 to 10:1, preferably 3:1 to 9:1, particularly 4:1 to 8:1. The use of the previously indicated weight ratios leads to particularly good antiperspirant effectiveness of the antiperspirant compound, which is negatively affected neither by incorporation into an aqueous phase of an O/W emulsion nor by long storage durations. Therefore, the amount of antiperspirant compound used can be reduced to improve the cosmetic properties.

The oil phase b) can include further active substances and ingredients. It has proven advantageous in the context of the present invention if the oil phase b) includes a cyclic dimethylsiloxane. The cosmetic properties of the O/W emulsions according to the invention can be further improved by additionally using this dimethylcyclosiloxane. It is therefore preferred in the context of the present invention if the oil phase b) additionally includes at least one dimethylcyclosiloxane in a total amount of 0.5 to 10 wt %, preferably 0.8 to 9.0 wt %, particularly 1.0 to 7.0 wt %, with respect to the total weight of the antiperspirant cosmetic agent, wherein the at least one dimethylcyclosiloxane has the formula (V),

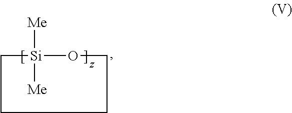

wherein
z represents integers from 2 to 10, preferably from 2 to 8, preferably from 2 to 6, particularly the integer 5.

It has also proven advantageous with regard to the cosmetic properties of the present invention if the oil phase b) additionally includes a specific alkane trisiloxane. Preferred embodiments of the present invention are therefore characterized in that the oil phase b) additionally includes 3-octylheptamethyltrisiloxane in a total amount of 1.0 to 12 wt %, preferably 2.0 to 10 wt %, particularly 3.0 to 9.0 wt %, with respect to the total weight of the antiperspirant cosmetic agent.

Furthermore, the oil phase b) can include a compound, selected from the group of linear, saturated alkanols having 8 to 30 carbon atoms, esters of alkanols with carboxylic acids, mono- and/or di- and/or triesters of glycerol with carboxylic acids, linear polydimethylsiloxanes, and mixtures thereof. In particular, the cosmetic properties of the O/W emulsions according to the invention can be adapted to the needs of the consumers or further improved by using such compounds. It is therefore preferred in the context of the present invention if the oil phase b) additionally includes at least one compound, selected from the group of (i) linear, saturated C₈-C₃₀-alkanols, (ii) esters of linear, saturated C₈-C₃₀-alkanols with C₈-C₃₀-carboxylic acids, (iii) mono- and/or di- and/or triesters of glycerol with linear, saturated C₈-C₃₀-carboxylic acids, (iv) linear polydimethylsiloxanes having a kinematic viscosity at 25° C. of 2 to 100 cSt, and (v) mixtures thereof. The kinematic viscosity of the linear polydimethylsiloxanes can be determined, for example, by means of the standard ASTM D445 (version from 1965).

In this context, it has proven advantageous if, as a linear, saturated C₈-C₃₀-alkanol, a linear, saturated C₂₂-alkanol is included in a total amount of 0.1 to 10 wt %, preferably 0.3 to 6.0 wt %, preferably 0.5 to 5.0 wt %, particularly 0.8 to 4.0 wt %, with respect to the total weight of the antiperspirant cosmetic agent. The use of behenyl alcohol has proven especially advantageous here.

In this context, it can also be advantageous according to the invention if, as an ester of linear, saturated C₈-C₃₀-alkanols with C₈-C₃₀-carboxylic acids, an ester of linear, saturated C₁₄-alkanols with C₁₄-carboxylic acids is included in a total amount of 0.1 to 10 wt %, preferably 0.5 to 7.0 wt %, preferably 0.8 to 5.0 wt %, particularly 1.0 to 4.0 wt %, with respect to the total weight of the antiperspirant cosmetic agent. An ester that is especially preferred according to the invention is myristyl myristate.

Furthermore, it has proven advantageous according to the invention if the antiperspirant O/W emulsion includes at least one mono- and/or di- and/or triester of glycerol with linear, saturated carboxylic acids. Preferred embodiments of the antiperspirant cosmetic agents according to the invention are therefore characterized in that, as a mono- and/or di- and/or triester of glycerol with linear, saturated C₈-C₃₀-carboxylic acids, a monoester of glycerol with linear saturated C₁₄-carboxylic acids is included in a total amount of 0.1 to 10 wt %, preferably 0.5 to 7.0 wt %, preferably 0.8 to 4.0 wt %, particularly 1.0 to 3.0 wt %, with respect to the total weight of the antiperspirant cosmetic agent. Compounds that are especially suitable in the context of this embodiment are monoesters of glycerol with linear, saturated C₁₄-carboxylic acids, such as glyceryl stearate.

Furthermore, it can be advantageous in this context if the antiperspirant O/W emulsion according to the invention includes at least one linear siloxane. It is therefore preferred according to the invention if, as a linear polydimethylsiloxane having a kinematic viscosity at 25° C. of 2 to 100 cSt, a linear polydimethylsiloxane having a kinematic viscosity at 25° C. of 2 to 10 cSt is included in a total amount of 3.0 to 45 wt %, preferably 5.0 to 40 wt %, preferably 8.0 to 30 wt %, particularly 10 to 22 wt %, with respect to the total weight of the antiperspirant cosmetic agent. A linear polydimethylsiloxane having a kinematic viscosity at 25° C. of 2 to 10 cSt is especially preferably used in the O/W emulsions according to the invention.

Preferred antiperspirant O/W emulsions according to the invention include the oil phase b) in certain amount ranges. Preferred embodiments of antiperspirant cosmetic agents according to the invention are therefore characterized in that the antiperspirant cosmetic agent includes the oil phase b) in a total amount of 15 to 50 wt %, preferably 20 to 40 wt %, preferably 25 to 40 wt %, particularly 28 to 40 wt %, with respect to the total weight of the antiperspirant cosmetic agent.

In the context of the present invention, it can be advantageous if the aqueous phase a) and/or the oil phase b) and/or the O/W emulsion according to the invention includes further active substances and ingredients.

In this context, it can be preferred if the O/W emulsion according to the invention includes at least one inorganic filler. Preferred embodiments of cosmetic agents according to the invention are therefore characterized in that the cosmetic agent additionally includes at least one inorganic filler selected from the group of silicon dioxide, silicic acids, spherical polyalkylsesquisiloxane particles, silica gels, talc, kaolin, bentonites, magnesium aluminum silicates, boron nitride, and mixtures thereof in a total amount of 0.5 to 8.0 wt %, preferably 1.0 to 7.0 wt %, preferably 1.5 to 6.0 wt %, particularly 2.0 to 5.0 wt %, with respect to the total weight of the cosmetic agent. Silicic acids, particularly pyrogenic silicic acids, and talc are especially preferably used.

In the context of the present invention, it can be preferred with regard to the stability of the O/W emulsion that at least one emulsifier is added. Emulsifiers are amphiphilic (bifunctional) compounds that consist of at least one hydrophobic molecule part and at least one hydrophilic molecule part. The hydrophobic molecule part is preferably a hydrocarbon chain having 8 to 28 carbon atoms, which hydrocarbon chain can be saturated or unsaturated, linear or branched. This C₈-C₂₈-alkyl chain is especially preferably linear.

Therefore, cosmetic agents are preferred which additionally include at least one emulsifier, selected from the group of alkoxylated C₈-C₂₄-alkanols having 2 to 30 moles of alkylene oxide per mole of alkanol, alkoxylated C₈-C₂₄-carboxylic acids having 2 to 30 moles of alkylene oxide per mole of carboxylic acid, silicone copolyols having ethylene oxide units or having ethylene oxide units and propylene oxide units, alkyl mono- and oligoglycosides having 8 to 22 carbon atoms in the alkyl residue and ethoxylated analogs thereof, ethoxylated sterols, partial esters of polyglycerols having 2 to 10 glycerol units and having 1 to 4 saturated or unsaturated, linear or branched C₈-C₂₂ fatty acid residues, in a total amount of 0.5 to 10 wt %, particularly 1.0 to 5.0 wt %, with respect to the total weight of the cosmetic agent. Especially preferred emulsifiers are ethoxylated C₈-C₂₄-alkanols having 2 to 30 moles of ethylene oxide per mole of alkanol and propoxylated C₈-C₂₄-alkanols having 10 to 20 moles of propylene oxide per mole of alkanol. In particular, stearyl ethers having 2 to 21 moles of ethylene oxide, stearyl ethers having 15 moles of propylene oxide, and cetearyl ethers having 12 to 30 moles of ethylene oxide are preferred in this context.

To further support the antiperspirant effect, it can be advantageous to add at least one chelating agent to the cosmetic agents according to the invention. Preferred cosmetic agents are therefore characterized in that they additionally include at least one chelating agent, selected from the group of ethylenediaminetetraacetic acid (EDTA) and salts thereof and nitrilotriacetic acid (NTA) and mixtures of these substances, in a total amount of 0.01 to 0.5 wt %, particularly 0.05 to 0.2 wt %, with respect to the total weight of the cosmetic agent.

Furthermore, the cosmetic agents can additionally include at least one cosmetic oil that is liquid at 20° C. and 1,013 hPa in the oil phase b). According to the invention, this term is understood to mean an oil that is suitable for cosmetic use and is not miscible with water. However, these oils are neither odorous substances nor ethereal oils. These cosmetic oils can be selected from the group of (i) volatile non-silicone oils, particularly liquid paraffin oils and isoparaffin oils, such as isodecane, isoundecane, isododecane, isotridecane, isotetradecane, isopentadecane, isohexadecane, and isoeicosane; (ii) non-volatile non-silicone oils, particularly the triethyl citrates, the dicarboxylic acid esters of linear or branched C₂-C₁₀-alkanols, the products of the addition of ethylene oxide and/or propylene oxide to mono- or polyhydric C₃₋₂₂-alkanols, which can optionally be esterified, the symmetrical, unsymmetrical, or cyclic esters of carbonic acid with fatty alcohols, the esters of dimers of unsaturated C₁₂₋₂₂ fatty acids with monohydric, linear, branched, and cyclic C₂₋₁₈-alkanols or C₂₋₆-alkanols, the benzoic acid esters of linear or branched C₈₋₂₂-alkanols, such as benzoic acid C₁₂₋₁₅-alkyl esters and benzoic acid isostearyl ester and benzoic acid octyldodecyl ester, the synthetic hydrocarbons, such as polyisobutylene and polydecene, the alicyclic hydrocarbons; and (iii) mixtures thereof.

According to the invention, the term “volatile cosmetic oil” refers to cosmetic oils that have a vapor pressure of 2.66 Pa to 40,000 Pa (0.02 to 300 mm Hg), preferably 10 to 12,000 Pa (0.1 to 90 mm Hg), more preferably 13 to 3,000 Pa (0.1 to 23 mm Hg), particularly 15 to 500 Pa (0.1 to 4 mm Hg), at 20° C. and an ambient pressure of 1,013 hPa. Furthermore, in the sense of the present invention, the term “non-volatile cosmetic oils” is understood to means cosmetic oils that have a vapor pressure of less than 2.66 Pa (0.02 mm Hg) at 20° C. and an ambient pressure of 1,013 hPa.

Also preferred according to the invention is the use of mixtures of the aforementioned cosmetic oils, particularly of non-volatile and volatile cosmetic oils, because in this way parameters such as skin feel, visibility of the residue, and stability of the cosmetic agent according to the invention can be set and the agent thus can be better adapted to the needs of the consumers.

In the context of the present invention, it is preferred if the cosmetic oil that is liquid at 20° C. and 1,013 hPa is included in a total amount of 1.0 to 50 wt %, preferably 2.0 to 40 wt %, preferably 3.0 to 30 wt %, more preferably 4.0 to 25 wt %, particularly 5.0 to 20 wt %, with respect to the total weight of the oil phase b).

The antiperspirant cosmetic agents according to the invention in the form of 0/W emulsions can be produced as follows. First, all constituents of the aqueous phase a) and of the oil phase b) are heated to 60 to 90° C. and then mixed while stirring is performed. After homogenization for 5 to 15 minutes, any further ingredients, such as inorganic fillers, are added and cooled to 20 to 25° C. while stirring is performed.

The use of certain antiperspirant compounds in combination with specific non-ionic cross-linked silicone polymers is especially advantageous with regard to the improved antiperspirant effect and improved cosmetic properties, particularly reduced tackiness and textile soiling. The embodiments AF 1 to AF 137 of the antiperspirant cosmetic agents according to the invention in the form of O/W emulsions, which are indicated in the tables below, are therefore especially preferred (all specifications in wt % and with respect to the total weight of the antiperspirant cosmetic agent according to the invention). In these embodiments AF 1 to AF 137, the weight ratio of the at least one antiperspirant compound, particularly of the aluminum sesquichlorohydrate, of the phosphate-containing aluminum-zirconium salt, and of the calcium-containing aluminum-zirconium salt, in the aqueous phase a) to the cross-linked silicone polymer in the oil phase b) is 4:1 to 12:1, preferably 3:1 to 11:1, preferably 3:1 to 10:1, more preferably 3:1 to 9:1, particularly 4:1 to 8:1.

| | AF 1 | AF 2 | AF 3 | AF 4 |
|---|---|---|---|---|
| Antiperspirant compound | 5.0-14 | 6.0-14 | 7.0-13 | 10-12 |
| Cross-linked silicone polymer | 0.4-5.0 | 0.5-4.5 | 0.6-4.0 | 0.8-3.5 |
| Oil phase b) | 15-50 | 20-40 | 25-40 | 28-40 |
| Water in aqueous phase a) | Ad 100 | Ad 100 | Ad 100 | Ad 100 |
| | AF 5 | AF 6 | AF 7 | AF 8 |
| Antiperspirant compound | 5.0-14 | 6.0-14 | 7.0-13 | 10-12 |
| Cross-linked silicone polymer ¹⁾ | 0.4-5.0 | 0.5-4.5 | 0.6-4.0 | 0.8-3.5 |
| Oil phase b) | 15-50 | 20-40 | 25-40 | 28-40 |
| Water in aqueous phase a) | Ad 100 | Ad 100 | Ad 100 | Ad 100 |
| | AF 9 | AF 10 | AF 11 | AF 12 |
| Aluminum sesquichloride | 5.0-14 | 6.0-14 | 7.0-13 | 10-12 |
| Cross-linked silicone polymer ²⁾ | 0.4-5.0 | 0.5-4.5 | 0.6-4.0 | 0.8-3.5 |
| Oil phase b) | 15-50 | 20-40 | 25-40 | 28-40 |
| Water in aqueous phase a) | Ad 100 | Ad 100 | Ad 100 | Ad 100 |
| | AF 13 | AF 14 | AF 15 | AF 16 |
| Aluminum sesquichloride | 5.0-14 | 6.0-14 | 7.0-13 | 10-12 |
| Cross-linked silicone polymer ³⁾ | 0.4-5.0 | 0.5-4.5 | 0.6-4.0 | 0.8-3.5 |
| Oil phase b) | 15-50 | 20-40 | 25-40 | 28-40 |
| Water in aqueous phase a) | Ad 100 | Ad 100 | Ad 100 | Ad 100 |
| | AF 17 | AF 18 | AF 19 | AF 20 |
| Phosphate-containing aluminum- | 5.0-14 | 6.0-14 | 7.0-13 | 10-12 |
| zirconium salt ⁴⁾ | | | | |
| Cross-linked silicone polymer ²⁾ | 0.4-5.0 | 0.5-4.5 | 0.6-4.0 | 0.8-3.5 |
| Oil phase b) | 15-50 | 20-40 | 25-40 | 28-40 |
| Water in aqueous phase a) | Ad 100 | Ad 100 | Ad 100 | Ad 100 |
| | AF 21 | AF 22 | AF 23 | AF 24 |
| Phosphate-containing aluminum- | 5.0-14 | 6.0-14 | 7.0-13 | 10-12 |
| zirconium salt ⁴⁾ | | | | |
| Cross-linked silicone polymer ³⁾ | 0.4-5.0 | 0.5-4.5 | 0.6-4.0 | 0.8-3.5 |
| Oil phase b) | 15-50 | 20-40 | 25-40 | 28-40 |
| Water in aqueous phase a) | Ad 100 | Ad 100 | Ad 100 | Ad 100 |
| | AF 25 | AF 26 | AF 27 | AF 28 |
| Calcium-containing aluminum- | 5.0-14 | 6.0-14 | 7.0-13 | 10-12 |
| zirconium salt ⁵⁾ | | | | |
| Cross-linked silicone polymer ²⁾ | 0.4-5.0 | 0.5-4.5 | 0.6-4.0 | 0.8-3.5 |
| Oil phase b) | 15-50 | 20-40 | 25-40 | 28-40 |
| Water in aqueous phase a) | Ad 100 | Ad 100 | Ad 100 | Ad 100 |
| | AF 29 | AF 30 | AF 31 | AF 32 |
| Calcium-containing aluminum- | 5.0-14 | 6.0-14 | 7.0-13 | 10-12 |
| zirconium salt ⁵⁾ | | | | |
| Cross-linked silicone polymer ³⁾ | 0.4-5.0 | 0.5-4.5 | 0.6-4.0 | 0.8-3.5 |
| Oil phase b) | 15-50 | 20-40 | 25-40 | 28-40 |
| Water in aqueous phase a) | Ad 100 | Ad 100 | Ad 100 | Ad 100 |
| | AF 33 | AF 34 | AF 35 | AF 36 |
| Phosphate-containing aluminum- | 5.0-14 | 6.0-14 | 7.0-13 | 10-12 |
| zirconium salt ⁶⁾ | | | | |
| Cross-linked silicone polymer ²⁾ | 0.4-5.0 | 0.5-4.5 | 0.6-4.0 | 0.8-3.5 |
| Oil phase b) | 15-50 | 20-40 | 25-40 | 28-40 |
| Water in aqueous phase a) | Ad 100 | Ad 100 | Ad 100 | Ad 100 |
| | AF 37 | AF 38 | AF 39 | AF 40 |
| Phosphate-containing aluminum- | 5.0-14 | 6.0-14 | 7.0-13 | 10-12 |
| zirconium salt ⁶⁾ | | | | |
| Cross-linked silicone polymer ³⁾ | 0.4-5.0 | 0.5-4.5 | 0.6-4.0 | 0.8-3.5 |
| Oil phase b) | 15-50 | 20-40 | 25-40 | 28-40 |
| Water in aqueous phase a) | Ad 100 | Ad 100 | Ad 100 | Ad 100 |
| | AF 41 | AF 42 | AF 43 | AF 44 |
| Calcium-containing aluminum- | 5.0-14 | 6.0-14 | 7.0-13 | 10-12 |
| zirconium salt ⁷⁾ | | | | |
| Cross-linked silicone polymer ²⁾ | 0.4-5.0 | 0.5-4.5 | 0.6-4.0 | 0.8-3.5 |
| Oil phase b) | 15-50 | 20-40 | 25-40 | 28-40 |
| Water in aqueous phase a) | Ad 100 | Ad 100 | Ad 100 | Ad 100 |
| | AF 45 | AF 46 | AF 47 | AF 48 |
| Calcium-containing aluminum- | 5.0-14 | 6.0-14 | 7.0-13 | 10-12 |
| zirconium salt ⁷⁾ | | | | |
| Cross-linked silicone polymer ³⁾ | 0.4-5.0 | 0.5-4.5 | 0.6-4.0 | 0.8-3.5 |
| Oil phase b) | 15-50 | 20-40 | 25-40 | 28-40 |
| Water in aqueous phase a) | Ad 100 | Ad 100 | Ad 100 | Ad 100 |
| | AF 49 | AF 50 | AF 51 | AF 52 |
| Antiperspirant compound | 5.0-14 | 6.0-14 | 7.0-13 | 10-12 |
| Cross-linked silicone polymer ¹⁾ | 0.4-5.0 | 0.5-4.5 | 0.6-4.0 | 0.8-3.5 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 53 | AF 54 | AF 55 | AF 56 |
| Aluminum sesquichloride | 5.0-14 | 6.0-14 | 7.0-13 | 10-12 |
| Cross-linked silicone polymer ²⁾ | 0.4-5.0 | 0.5-4.5 | 0.6-4.0 | 0.8-3.5 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 57 | AF 58 | AF 59 | AF 60 |
| Aluminum sesquichloride | 5.0-14 | 6.0-14 | 7.0-13 | 10-12 |
| Cross-linked silicone polymer ³⁾ | 0.4-5.0 | 0.5-4.5 | 0.6-4.0 | 0.8-3.5 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 61 | AF 62 | AF 63 | AF 64 |
| Phosphate-containing aluminum- | 5.0-14 | 6.0-14 | 7.0-13 | 10-12 |
| zirconium salt ⁴⁾ | | | | |
| Cross-linked silicone polymer ²⁾ | 0.4-5.0 | 0.5-4.5 | 0.6-4.0 | 0.8-3.5 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 65 | AF 66 | AF 67 | AF 68 |
| Phosphate-containing aluminum- | 5.0-14 | 6.0-14 | 7.0-13 | 10-12 |
| zirconium salt ⁴⁾ | | | | |
| Cross-linked silicone polymer ³⁾ | 0.4-5.0 | 0.5-4.5 | 0.6-4.0 | 0.8-3.5 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 70 | AF 71 | AF 72 | AF 73 |
| Calcium-containing aluminum- | 5.0-14 | 6.0-14 | 7.0-13 | 10-12 |
| zirconium salt ⁵⁾ | | | | |
| Cross-linked silicone polymer ²⁾ | 0.4-5.0 | 0.5-4.5 | 0.6-4.0 | 0.8-3.5 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 74 | AF 75 | AF 76 | AF 77 |
| Calcium-containing aluminum- | 5.0-14 | 6.0-14 | 7.0-13 | 10-12 |
| zirconium salt ⁵⁾ | | | | |
| Cross-linked silicone polymer ³⁾ | 0.4-5.0 | 0.5-4.5 | 0.6-4.0 | 0.8-3.5 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 78 | AF 79 | AF 80 | AF 81 |
| Phosphate-containing aluminum- | 5.0-14 | 6.0-14 | 7.0-13 | 10-12 |
| zirconium salt ⁶⁾ | | | | |
| Cross-linked silicone polymer ²⁾ | 0.4-5.0 | 0.5-4.5 | 0.6-4.0 | 0.8-3.5 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 82 | AF 83 | AF 84 | AF 85 |
| Phosphate-containing aluminum- | 5.0-14 | 6.0-14 | 7.0-13 | 10-12 |
| zirconium salt ⁶⁾ | | | | |
| Cross-linked silicone polymer ³⁾ | 0.4-5.0 | 0.5-4.5 | 0.6-4.0 | 0.8-3.5 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 86 | AF 87 | AF 88 | AF 89 |
| Calcium-containing aluminum- | 5.0-14 | 6.0-14 | 7.0-13 | 10-12 |
| zirconium salt ⁷⁾ | | | | |
| Cross-linked silicone polymer ²⁾ | 0.4-5.0 | 0.5-4.5 | 0.6-4.0 | 0.8-3.5 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 90 | AF 91 | AF 92 | AF 93 |
| Calcium-containing aluminum- | 5.0-14 | 6.0-14 | 7.0-13 | 10-12 |
| zirconium salt ⁷⁾ | | | | |
| Cross-linked silicone polymer ³⁾ | 0.4-5.0 | 0.5-4.5 | 0.6-4.0 | 0.8-3.5 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 94 | AF 95 | AF 96 | AF 97 |
| Antiperspirant compound | 5.0-14 | 6.0-14 | 7.0-13 | 10-12 |
| Cross-linked silicone polymer ¹⁾ | 0.4-5.0 | 0.5-4.5 | 0.6-4.0 | 0.8-3.5 |
| Non-ionic associative polymer ⁹⁾ | 0.01-4.0 | 0.1-3.0 | 0.2-2.0 | 0.3-1.0 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 98 | AF 99 | AF 100 | AF 101 |
| Aluminum sesquichloride | 5.0-14 | 6.0-14 | 7.0-13 | 10-12 |
| Cross-linked silicone polymer ²⁾ | 0.4-5.0 | 0.5-4.5 | 0.6-4.0 | 0.8-3.5 |
| Non-ionic associative polymer ⁹⁾ | 0.01-4.0 | 0.1-3.0 | 0.2-2.0 | 0.3-1.0 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 102 | AF 103 | AF 104 | AF 105 |
| Aluminum sesquichloride | 5.0-14 | 6.0-14 | 7.0-13 | 10-12 |
| Cross-linked silicone polymer ³⁾ | 0.4-5.0 | 0.5-4.5 | 0.6-4.0 | 0.8-3.5 |
| Non-ionic associative polymer ⁹⁾ | 0.01-4.0 | 0.1-3.0 | 0.2-2.0 | 0.3-1.0 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 106 | AF 107 | AF 108 | AF 109 |
| Phosphate-containing aluminum- | 5.0-14 | 6.0-14 | 7.0-13 | 10-12 |
| zirconium salt ⁴⁾ | | | | |
| Cross-linked silicone polymer ²⁾ | 0.4-5.0 | 0.5-4.5 | 0.6-4.0 | 0.8-3.5 |
| Non-ionic associative polymer ⁹⁾ | 0.01-4.0 | 0.1-3.0 | 0.2-2.0 | 0.3-1.0 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 110 | AF 111 | AF 112 | AF 113 |
| Phosphate-containing aluminum- | 5.0-14 | 6.0-14 | 7.0-13 | 10-12 |
| zirconium salt ⁴⁾ | | | | |
| Cross-linked silicone polymer ³⁾ | 0.4-5.0 | 0.5-4.5 | 0.6-4.0 | 0.8-3.5 |
| Non-ionic associative polymer ⁹⁾ | 0.01-4.0 | 0.1-3.0 | 0.2-2.0 | 0.3-1.0 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 114 | AF 115 | AF 116 | AF 117 |
| Calcium-containing aluminum- | 5.0-14 | 6.0-14 | 7.0-13 | 10-12 |
| zirconium salt ⁵⁾ | | | | |
| Cross-linked silicone polymer ²⁾ | 0.4-5.0 | 0.5-4.5 | 0.6-4.0 | 0.8-3.5 |
| Non-ionic associative polymer ⁹⁾ | 0.01-4.0 | 0.1-3.0 | 0.2-2.0 | 0.3-1.0 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 118 | AF 119 | AF 120 | AF 121 |
| Calcium-containing aluminum- | 5.0-14 | 6.0-14 | 7.0-13 | 10-12 |
| zirconium salt ⁵⁾ | | | | |
| Cross-linked silicone polymer ³⁾ | 0.4-5.0 | 0.5-4.5 | 0.6-4.0 | 0.8-3.5 |
| Non-ionic associative polymer ⁹⁾ | 0.01-4.0 | 0.1-3.0 | 0.2-2.0 | 0.3-1.0 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 122 | AF 123 | AF 124 | AF 125 |
| Phosphate-containing aluminum- | 5.0-14 | 6.0-14 | 7.0-13 | 10-12 |
| zirconium salt ⁶⁾ | | | | |
| Cross-linked silicone polymer ²⁾ | 0.4-5.0 | 0.5-4.5 | 0.6-4.0 | 0.8-3.5 |
| Non-ionic associative polymer ⁹⁾ | 0.01-4.0 | 0.1-3.0 | 0.2-2.0 | 0.3-1.0 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 126 | AF 127 | AF 128 | AF 129 |
| Phosphate-containing aluminum- | 5.0-14 | 6.0-14 | 7.0-13 | 10-12 |
| zirconium salt ⁶⁾ | | | | |
| Cross-linked silicone polymer ³⁾ | 0.4-5.0 | 0.5-4.5 | 0.6-4.0 | 0.8-3.5 |
| Non-ionic associative polymer ⁹⁾ | 0.01-4.0 | 0.1-3.0 | 0.2-2.0 | 0.3-1.0 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 130 | AF 131 | AF 132 | AF 133 |
| Calcium-containing aluminum- | 5.0-14 | 6.0-14 | 7.0-13 | 10-12 |
| zirconium salt ⁷⁾ | | | | |
| Cross-linked silicone polymer ²⁾ | 0.4-5.0 | 0.5-4.5 | 0.6-4.0 | 0.8-3.5 |
| Non-ionic associative polymer ⁹⁾ | 0.01-4.0 | 0.1-3.0 | 0.2-2.0 | 0.3-1.0 |
| Oil phase b) ⁸⁾ | 15-50 | 20-40 | 25-40 | 28-40 |
| Water in aqueous phase a) | Ad 100 | Ad 100 | Ad 100 | Ad 100 |
| | AF 134 | AF 135 | AF 136 | AF 137 |
| Calcium-containing aluminum- | 5.0-14 | 6.0-14 | 7.0-13 | 10-12 |
| zirconium salt ⁷⁾ | | | | |
| Cross-linked silicone polymer ³⁾ | 0.4-5.0 | 0.5-4.5 | 0.6-4.0 | 0.8-3.5 |
| Non-ionic associative polymer ⁹⁾ | 0.01-4.0 | 0.1-3.0 | 0.2-2.0 | 0.3-1.0 |
| Oil phase b) ⁸⁾ | 15-50 | 20-40 | 25-40 | 28-40 |
| Water in aqueous phase a) | Ad 100 | Ad 100 | Ad 100 | Ad 100 |

¹⁾ cross-linked silicone polymer selected from the group of (i) non-ionic cross-linked silicone polymers having at least the structural units of formulas (Ia) and (IIa) and (IIIa) above; (ii) non-ionic cross-linked silicone polymers having at least the structural units of formulas (Ib) and (IIb) and (IIIb) above; (iii) non-ionic cross-linked silicone polymers having at least the structural units of formulas (Ic) and (IIc) and (IIIc) above; (iv) cationic cross-linked silicone polymers having at least the structural units of formulas (Id) and (IId) and (IIId) above; and (v) mixtures thereof,
²⁾ non-ionic cross-linked silicone polymer having at least the structural units of formulas (Ia) and (IIa) and (IIIa) above, wherein n represents the integer 6 in formula (IIIa),
³⁾ non-ionic cross-linked silicone polymer having at least the structural units of formulas (Ib) and (IIb) and (IIIb) above, wherein m represents integers from 10 to 14 in formula (IIIb) and o represents integers from 18 to 22 in formula (IVb),
⁴⁾ the phosphate-containing aluminum-zirconium salt used has the following parameters: total amount of phosphate ions (PO₄³⁻) is 0.1 to 0.3 wt %, with respect to the total weight of the phosphate-containing aluminum-zirconium salt, molar ratio of phosphate ions (PO₄³⁻) to aluminum is 0.02:1 to 0.151:1, and molar ratio of the sum of aluminum and zirconium to chloride is 0.9:1 to 2.1:1,
⁵⁾ the calcium-containing aluminum-zirconium salt used has the following parameters: total amount of calcium ions (Ca²⁺) is 0.4 to 0.7 wt %, with respect to the total weight of the calcium-containing aluminum-zirconium salt, molar ratio of the sum of aluminum and zirconium to chloride is 0.9:1 to 2.1:1,
⁶⁾ the phosphate-containing aluminum-zirconium salt used has the following parameters: total amount of phosphate ions (PO₄³⁻) is 0.1 to 0.3 wt % and total amount of glycerol is 4.0 to 6.0 wt %, in each case with respect to the total weight of the phosphate-containing aluminum-zirconium salt, molar ratio of phosphate ions (PO₄³⁻) to aluminum is 0.02:1 to 0.151:1, and molar ratio of the sum of aluminum and zirconium to chloride is 0.9:1 to 2.1:1,
⁷⁾ the calcium-containing aluminum-zirconium salt used has the following parameters: total amount of calcium ions (Ca²⁺) is 0.4 to 0.7 wt % and total amount of glycerol is 4.0 to 6.0 wt %, in each case with respect to the total weight of the calcium-containing aluminum-zirconium salt, molar ratio of the sum of aluminum and zirconium to chloride is 0.9:1 to 2.1:1,
⁸⁾ oil phase includes or consists of, in each case with respect to the total weight of the cosmetic agent, 0.8 to 4.0 wt % of a linear, saturated C₂₂-alkanol, 1.0 to 4.0 wt % of an ester of a linear saturated C₁₄-alkanol with a C₁₄-carboxylic acid, 1.0 to 3.0 wt % of a monoester of glycerol with a linear saturated C₁₄-carboxylic acid, and 10 to 22 wt % of a linear polydimethylsiloxane having a kinematic viscosity at 25° C. of 2 to 10 cSt
⁹⁾ steareth-100/PEG-136/HDI copolymer.

The previously indicated embodiments 1 to 137 of the antiperspirant cosmetic agents according to the invention in the form of O/W emulsions have an excellent antiperspirant effect. By adding the cross-linked silicone polymer, the amount of antiperspirant compound, particularly aluminum sesquichlorohydrate, phosphate-containing aluminum-zirconium salt, and calcium-containing aluminum-zirconium salt, can be lowered to achieve an antiperspirant effect comparable to antiperspirant cosmetic agents without cross-linked silicone polymers. The reduction of the amount of antiperspirant compound, particularly aluminum sesquichlorohydrate, phosphate-containing aluminum-zirconium salt, and calcium-containing aluminum-zirconium salt, leads to improved cosmetic properties, particularly reduced tackiness, reduced staining of textiles, and improved skin compatibility.

The antiperspirant cosmetic agent according to the invention can be applied by means of various methods. According to a preferred embodiment, the cosmetic agent is formulated as a cream application. Furthermore, it is also possible to apply the cosmetic agents according to the invention in the form of a solid emulsion by means of a solid stick. Therefore, it is preferred according to the invention if the cosmetic agent is formulated as a stick application.

However, it can also be preferred according to the invention that the antiperspirant cosmetic agent is included on and/or in a disposable substrate, selected from the group of wipes, pads, and puffs. Especially preferred are wet wipes, i.e., preferably individually packaged wet wipes prefabricated for the user, which are well known, for example, from the field of glass cleaning or from the field of wet toilet wipes. Such wet wipes, which can advantageously also include preservatives, are impregnated or loaded with a cosmetic agent according to the invention and are preferably packaged individually. They can be used, for example, as a deodorant wipe, which is particularly of interest for use on the go. Preferred substrate materials are selected from porous flat wipes. They can consist of a fibrous or cellular flexible material that has sufficient mechanical stability and at the same time softness for use on the skin. These wipes include wipes composed of woven and nonwoven (fleece) synthetic and natural fibers, felt, paper, or foam, such as hydrophilic polyurethane foam. Deodorizing or antiperspirant substrates preferred according to the invention can be obtained by soaking or impregnation or by applying a cosmetic agent according to the invention to a substrate in melted form.

The present invention also relates to a non-therapeutic cosmetic method for preventing and/or reducing the perspiration of the body, wherein a cosmetic agent according to the invention is applied to the skin, particularly to the skin of the armpits, and remains on the skin for at least 1 hour, preferably for at least 2 hours, preferably for at least 4 hours, particularly for at least 6 hours.

The method according to the invention using the cosmetic agents according to the invention in the form of O/W emulsions, which cosmetic agent is a combination of antiperspirant compound with at least one cross-linked silicone polymer, achieves, with a reduced amount of antiperspirant compound, a comparable antiperspirant effect in comparison with antiperspirants of the prior art that do not have the previously indicated combination. Because of the smaller amount of antiperspirant compound used in the method according to the invention, improved cosmetic properties are achieved, particularly reduced tackiness, reduced staining of textiles, and improved skin compatibility.

That which was said with regard to the cosmetic agents according to the invention applies, mutatis mutandis, to additional preferred embodiments of the method according to the invention, particularly to the cosmetic agents used there.

Finally, the present invention also relates to the use of at least one cross-linked silicone polymer to improve the antiperspirant effect of O/W emulsions that include at least one antiperspirant compound, selected from the group of aluminum sesquichlorohydrate, phosphate-containing aluminum-zirconium salts, calcium-containing aluminum-zirconium salts, and mixtures thereof, in an aqueous phase. By adding the cross-linked silicone polymer, an improved antiperspirant effect of the O/W emulsions in comparison with O/W emulsions without cross-linked silicone polymers is obtained. As a result, the amount of antiperspirant compound can be reduced to achieve improved cosmetic properties, such as reduced tackiness and textile soiling and improved skin compatibility.

According to a preferred embodiment of this subject matter of the invention, at least one non-ionic cross-linked silicone polymer having at least one structural unit of formula (Ia) and at least one structural unit of formula (IIa) and at least one structural unit of formula (IIIa),

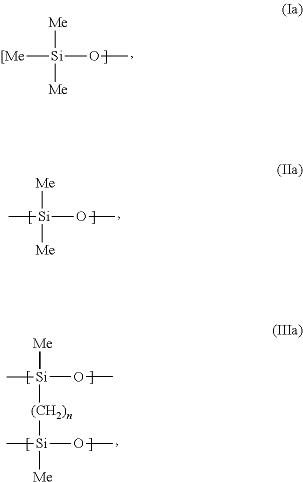

wherein

n represents the integer 6, includes, is used as a cross-linked silicone polymer.

According to another preferred embodiment of this subject matter of the invention, at least one non-ionic cross-linked silicone polymer having at least one structural unit of formula (Ib) and at least one structural unit of formula (IIb) and at least one structural unit of formula (IIIb) and at least one structural unit of formula (IVb),

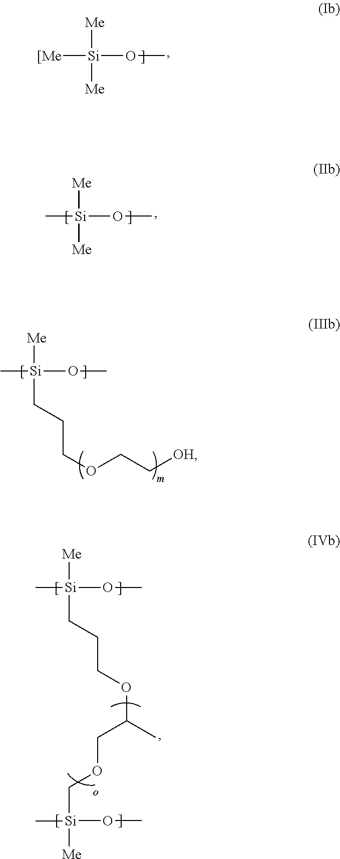

wherein

m represents integers from 10 to 14 and o represents integers from 18 to 22, includes, is used as a cross-linked silicone polymer.

That which was said with regard to the antiperspirant cosmetic agent according to the invention and the method according to the invention applies, mutatis mutandis, to additional embodiments of the use according to the invention, particularly to the cross-linked silicone polymers used and the further constituents of the O/W emulsions.

The following examples illustrate the present invention without restricting the present invention thereto:

### Examples

In the examples below, phosphate-containing aluminum-zirconium salts that have a total amount of phosphate ions (PO₄³⁻) of 0.1 to 0.3 wt % and a total amount of glycerol of 4.0 to 6.0 wt %, in each case with respect to the total weight of the phosphate-containing aluminum-zirconium salt, a molar ratio of phosphate ions (PO₄³⁻) to aluminum of 0.02:1 to 0.151:1, and a molar ratio of the sum of aluminum and zirconium to chloride of 0.9:1 to 2.1:1 are preferably used.

Furthermore, calcium-containing aluminum-zirconium salts that have a total amount of calcium ions (Ca²⁺) of 0.4 to 0.7 wt % and a total amount of glycerol of 4.0 to 6.0 wt %, in each case with respect to the total weight of the calcium-containing aluminum-zirconium salt, and a molar ratio of the sum of aluminum and zirconium to chloride of 0.9:1 to 2.1:1 are preferably used in these examples.

The amount specifications below are indicated in wt %, with respect to the total weight of the respective cosmetic agents according to the invention.

| Phase | Raw substance | 1.1 | 1.2 | 1.3 | 1.4 |
|---|---|---|---|---|---|
| a) | Aluminum | 10 | — | — | — |
| | sesquichlorohydrate | | | | |
| | Phosphate-containing | — | 10 | — | 12 |
| | aluminum-zirconium salt | | | | |
| | Calcium-containing | — | — | 8.4 | — |
| | aluminum-zirconium salt | | | | |
| | Steareth-100/PEG-136/HDI | 1.0 | 1.0 | 1.0 | 1.0 |
| | copolymer | | | | |
| | Water | 61.2 | 61.2 | 62.8 | 59.2 |
| b) | Cross-linked silicone polymer ^{I)} | — | — | — | 1.8 |
| | Cross-linked silicone polymer ^{II)} | 1.8 | 1.8 | 1.8 | — |
| | Behenyl alcohol | 2.0 | 2.0 | 2.0 | 2.0 |
| | Myristyl myristate | 2.0 | 2.0 | 2.0 | 2.0 |
| | Glyceryl stearate | 2.0 | 2.0 | 2.0 | 2.0 |
| | Polydimethylsiloxane, 5 cSt | 20.0 | 20.0 | 20.0 | 20.0 |

^{I)} non-ionic silicone polymer having structural units of formulas (Ia) and (IIa) and (IIIa), wherein n represents the integer 6 in formula (IIIa),
^{II)} non-ionic silicone polymer having structural units of formulas (IIa) and (IIb) and (IIIb) and (IVb), wherein m represents integers from 10 to 14 in formula (IIIb) and o represents integers from 18 to 22 in formula (IVb)

To produce the antiperspirant cosmetic agents according to the invention in the form of an O/W emulsion, the aqueous phase a) and the oil phase b) are first heated to 80° C. separately and mixed while stirring is performed. After homogenization for 10 minutes, cooling to 20 to 25° C. is performed slowly while stirring is performed.

The agents 1.1 to 1.4 according to the invention have an excellent antiperspirant effect and cosmetic properties, particularly reduced tackiness.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant cosmetic agent in the form of an oil-in-water (O/W) emulsion, containing
a) at least one aqueous phase, comprising at least one antiperspirant compound, selected from aluminum sesquichlorohydrate, phosphate-containing aluminum-zirconium salts, calcium-containing aluminum-zirconium salts, and mixtures thereof,
b) at least one oil phase, comprising at least one cross-linked silicone polymer, wherein the weight ratio of the at least one antiperspirant compound in the aqueous phase to the at least one cross-linked silicone polymer in the oil phase is 3:1 to 12:1.

2. The antiperspirant cosmetic agent according to claim 1, wherein the at least one phosphate-containing aluminum-zirconium salt includes 0.01 to 1.0 wt % phosphate with respect to the total weight of the phosphate-containing aluminum-zirconium salt.

3. The antiperspirant cosmetic agent according to claim 1, wherein the at least one phosphate-containing aluminum-zirconium salt includes 0.07 to 0.5 wt % phosphate with respect to the total weight of the phosphate-containing aluminum-zirconium salt.

4. The antiperspirant cosmetic agent according to claim 1, wherein the at least one phosphate-containing aluminum-zirconium salt has a molar ratio of phosphate, to zirconium of 0.001:1 to 0.5:1.

5. The antiperspirant cosmetic agent according to claim 1, wherein the at least one phosphate-containing aluminum-zirconium salt has a molar ratio of phosphate, to zirconium of 0.01:1 to 0.3:1.

6. The antiperspirant cosmetic agent according to claim 1, wherein the at least one phosphate-containing and/or calcium-containing aluminum-zirconium salt has a molar ratio of the sum of aluminum and zirconium (Al+Zr) to chloride of 0.3:1 to 2.5:1.

7. The antiperspirant cosmetic agent according to claim 1, wherein the at least one phosphate-containing and/or calcium-containing aluminum-zirconium salt has a molar ratio of the sum of aluminum and zirconium (Al+Zr) to chloride 0.9:1 to 2.1:1.

8. The antiperspirant cosmetic agent according to claim 1, wherein the at least one phosphate-containing and/or calcium-containing aluminum-zirconium salt additionally includes glycine in a total amount of 2.0 to 8.0 wt % with respect to the total weight of the phosphate-containing or calcium-containing antiperspirant aluminum-zirconium salt.

9. The antiperspirant cosmetic agent according to claim 1, wherein the at least one calcium-containing aluminum-zirconium salt includes calcium in a total amount of 0.1 to 6.0 wt % with respect to the total weight of the calcium-containing aluminum-zirconium salt.

10. The antiperspirant cosmetic agent according to claim 1, wherein the antiperspirant cosmetic agent includes the at least one antiperspirant compound in a total amount of 5.0 to 14 wt % with respect to the total weight of the antiperspirant cosmetic agent.

11. The antiperspirant cosmetic agent according to claim 1, wherein the aqueous phase a) includes water in a total amount of 55 to 94 wt % with respect to the total weight of the aqueous phase a).

12. The antiperspirant cosmetic agent according to claim 1, wherein the antiperspirant cosmetic agent includes the aqueous phase a) in a total amount of 60 to 90 wt % with respect to the total weight of the antiperspirant cosmetic agent.

13. The cosmetic agent according to claim 1, wherein the aqueous phase a) additionally includes in a total amount of 0.01 to 4.0 wt % at least one non-ionic associative polymer with respect to the total weight of the aqueous phase a).

14. The cosmetic agent according to claim 1, wherein the aqueous phase a) additionally includes in a total amount of 0.3 to 1.0 wt % at least one non-ionic associative polymer with respect to the total weight of the aqueous phase a).

15. The cosmetic agent according to claim 13, wherein the non-ionic associative polymer is a steareth-100/PEG-136/HDI copolymer.

16. The antiperspirant cosmetic agent to claim 1, wherein the cross-linked silicone polymer is selected from the group of non-ionic cross-linked silicone polymers, cationic cross-linked silicone polymers, and mixtures thereof.

17. The antiperspirant cosmetic agent according to claim 16, wherein cosmetic agent includes at least one cross-linked silicone polymer having at least one structural unit of formula (Ia) and at least one structural unit of formula (IIa) and at least one structural unit of formula (IIIa) is included as a non-ionic cross-linked silicone polymer, 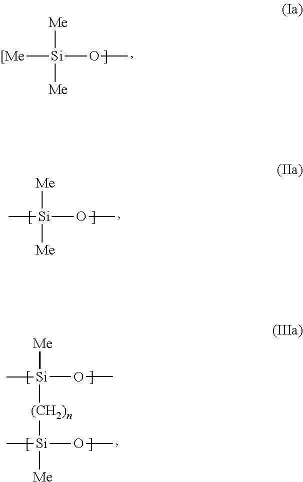
wherein
n represents integers from 2 to 10, preferably from 4 to 8, particularly 6.

18. The antiperspirant cosmetic agent according to claim 16, wherein the cosmetic agent includes at least one cross-linked silicone polymer having at least one structural unit of formula (Ib) and at least one structural unit of formula (IIb) and at least one structural unit of formula (IIIb) and at least one structural unit of formula (IVb) is included as a non-ionic cross-linked silicone polymer, 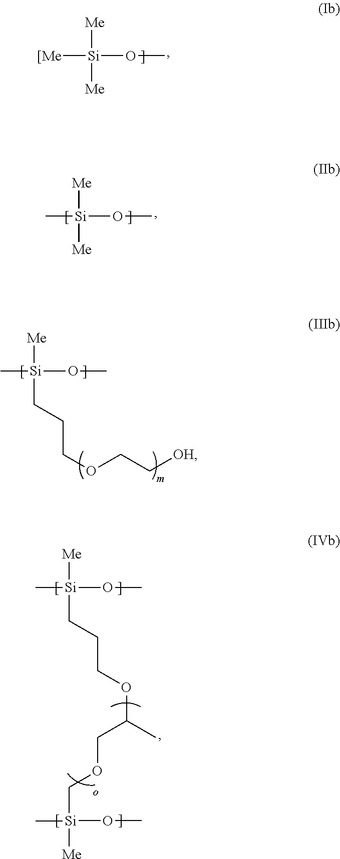
wherein
m represents integers from 6 to 16, preferably from 8 to 14, particularly from 10 to 14, and
o represents integers from 10 to 30, preferably from 14 to 26, particularly from 18 to 22.

19. The antiperspirant cosmetic agent according to claim 16, wherein the antiperspirant cosmetic agent includes a cross-linked silicone polymer having at least one structural unit of formula (Ic) and at least one structural unit of formula (IIc) and at least one structural unit of formula (IIIc) is included as a non-ionic cross-linked silicone polymer, 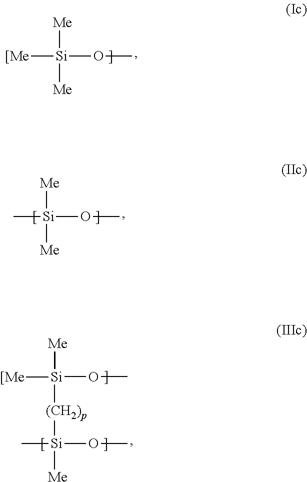
wherein
p represents integers from 2 to 6, preferably from 2 to 4, particularly 2.

20. The antiperspirant cosmetic agent according to claim 16, wherein the antiperspirant cosmetic agent includes a cross-linked silicone polymer having at least one structural unit of formula (Id) and at least one structural unit of formula (IId) and at least one structural unit of formula (IIId) and at least one structural unit of formula (IVd) is included as a cationic cross-linked silicone polymer, 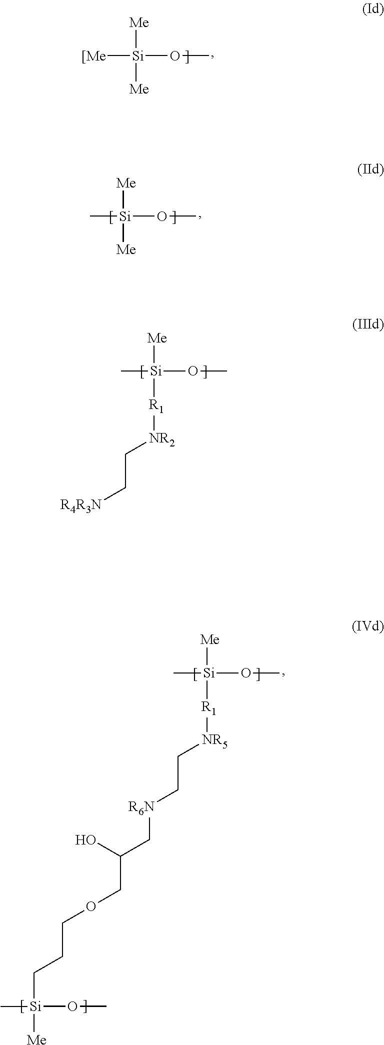
wherein
R₁ represents —CH₂—CH(CH₃)—CH₂— or —C(CH(CH₃)₂)—,
R₂ to R₄ represent, independently of each other, hydrogen or —CH₂—CH(OH)—CH₂—N⁺(Me)₃Cl⁻, and
R₅ and R₆ represent, independently of each other in each case, hydrogen, —CH₂—CH(OH)—CH₂—N⁺(Me)₃Cl⁻, or —CH₂—CH(OH)—CH₂—O—(CH₂)₃—Si(Me)O_{2/2}.

21. The antiperspirant cosmetic agent according to claim 1, wherein the cross-linked silicone polymer has an average particle size D₅₀ of 0.2 to 20 μm.

22. The antiperspirant cosmetic agent according to claim 1, wherein the antiperspirant cosmetic agent includes the at least one cross-linked silicone polymer in a total amount of 0.4 to 5.0 wt % with respect to the total weight of the antiperspirant cosmetic agent.

23. The antiperspirant cosmetic agent according to claim 1, wherein the antiperspirant cosmetic agent has a weight ratio of the at least one antiperspirant compound in the aqueous phase a) to the cross-linked silicone polymer in the oil phase b) of 3:1 to 11:1.

24. The antiperspirant cosmetic agent according to claim 1, wherein the oil phase b) additionally includes at least one dimethylcyclosiloxane in a total amount of 0.5 to 10 wt % with respect to the total weight of the antiperspirant cosmetic agent, and wherein the at least one dimethylcyclosiloxane has the formula (V), 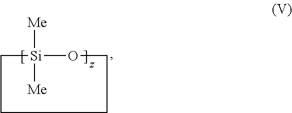
wherein
z represents integers from 2 to 10, preferably from 2 to 8, preferably from 2 to 6, particularly the integer 5.

25. The antiperspirant cosmetic agent according to claim 1, wherein the oil phase b) additionally includes 3-octylheptamethyltrisiloxane in a total amount of 1.0 to 12 wt % with respect to the total weight of the antiperspirant cosmetic agent.

26. The antiperspirant cosmetic agent according to claim 1, wherein the oil phase b) additionally includes at least one compound, selected from the group consisting of: (i) linear, saturated C₈-C₃₀-alkanols, (ii) esters of linear, saturated C₈-C₃₀-alkanols with C₈-C₃₀-carboxylic acids, (iii) mono- and/or di- and/or triesters of glycerol with linear, saturated C₈-C₃₀-carboxylic acids, (iv) linear polydimethylsiloxanes having a kinematic viscosity at 25° C. of 2 to 100 cSt, and (v) mixtures thereof.

27. The antiperspirant cosmetic agent according to claim 26, wherein the antiperspirant cosmetic agent includes 0.1 to 10 wt %, with respect to the total weight of the antiperspirant cosmetic agent, of a linear, saturated C₈-C₃₀-alkanol, a linear, saturated C₂₂-alkanol.

28. The antiperspirant cosmetic agent according to claim 26, wherein the antiperspirant cosmetic agent includes 0.1 to 10 wt %, with respect to the total weight of the antiperspirant cosmetic agent, of an ester of linear, saturated C₈-C₃₀-alkanols with C₈-C₃₀-carboxylic acids.

29. The antiperspirant cosmetic agent according to claim 28, wherein the antiperspirant cosmetic agent includes 0.1 to 10 wt %, with respect to the total weight of the antiperspirant cosmetic agent, of an ester of linear, saturated C₁₄-alkanols with C₁₄-carboxylic acids.

30. The antiperspirant cosmetic agent according to claim 26, wherein the antiperspirant cosmetic agent includes 0.1 to 10 wt %, with respect to the total weight of the antiperspirant cosmetic agent, of a mono- and/or di- and/or triester of glycerol with linear, saturated C₈-C₃₀-carboxylic acids with respect to the total weight of the antiperspirant cosmetic agent.

31. The antiperspirant cosmetic agent according to claim 26, wherein the antiperspirant cosmetic agent includes 3.0 to 45 wt %, with respect to the total weight of the antiperspirant cosmetic agent, of a linear polydimethylsiloxane having a kinematic viscosity at 25° C. of 2 to 100 cSt.

32. The antiperspirant cosmetic agent according to claim 1, wherein the antiperspirant cosmetic agent includes the oil phase b) in a total amount of 15 to 50 wt % with respect to the total weight of the antiperspirant cosmetic agent.
